(19) United States
(12) Patent Application Publication
(10) Pub. No.: US 2013/0109082 A1
(43) Pub. Date: May 2, 2013

(54) DEVICE FOR SEQUENTIALLY DISPENSING LIQUID REAGENTS TO A REACTION CHAMBER

(71) Applicants: Edward John Quinn, Wetherby (GB); Simon Webster, Wetherby (GB); David Alistair Smith, Wetherby (GB); Robert Harrand, Wetherby (GB)

(72) Inventors: Edward John Quinn, Wetherby (GB); Simon Webster, Wetherby (GB); David Alistair Smith, Wetherby (GB); Robert Harrand, Wetherby (GB)

(73) Assignee: AVACTA LIMITED, Wetherby (GB)

(21) Appl. No.: 13/639,790

(22) PCT Filed: Apr. 8, 2011
(86) PCT No.: PCT/GB11/00545

§ 371 Date: Dec. 26, 2012

(30) Foreign Application Priority Data
Apr. 8, 2010 (GB) GB1005885.7

(51) Int. Cl. B01L 3/02; G01N 33/50; C12M 1/34; G01N 31/00

(52) U.S. Cl. 435/287.2; 422/522; 422/81; 422/430

(57) ABSTRACT

An apparatus for delivering a plurality of liquid volumes sequentially to a reaction site is provided, the apparatus comprising a vessel having a plurality of solid spacers arranged in linear order therein so as to define a series of adjacent voids, separated by individual solid spacers, for receiving said liquid volumes, the vessel being provided in a lateral wall with a plurality of reversibly sealable openings through which the liquid volumes for delivery to the reaction site may be introduced into the voids defined therein, and an outlet port, the spacers being movable within the vessel such that, in use, liquid volumes disposed in the individual voids are deliverable sequentially through the ‘outlet port to the reaction site. Also provided are units, such as disposable cartridge units, comprising such an apparatus coupled to a reaction site and systems comprising such units together with means for interrogating the reaction site to determine the outcome of the reaction.

### FIELD OF THE INVENTION

The present invention relates to apparatus and systems for use in performing biological or chemical reactions and also to units, in particular disposable units, for use in such systems, and methods for assaying biological or chemical samples using such apparatus and units.

### BACKGROUND TO THE INVENTION

The delivery of liquid reagents, solvents or washes to a reaction site for use in a chemical or biological reaction is a requirement in many chemical or biological sensing, analytical and diagnostic applications.

Conventionally, the cost and complexity of the equipment required to perform such biological and chemical assays, together with the risk of contamination and cross-contamination, has meant that samples have generally been taken in the field and then transferred to a laboratory for analysis.

However, this introduces delay and is particularly undesirable in the case of assays for the diagnosis and treatment of human and animal diseases. There has therefore been considerable interest in the development of sensing, analytical and diagnostic techniques which can be delivered at the point-of-sampling or point-of-care and which can provide results more rapidly.

Diagnostic applications typically require a clinical sample such as blood, urine or saliva to be mixed with other reagents in liquid form to carry out a biological or chemical reaction, the interaction between the reagents being detectable, for example, in the form of a colour change detected using absorbance measurements or light detection methods such as luminescence and fluorescence. A number of technologies have been developed to deal with the challenge of providing smaller, low-cost devices in order to enable the mixing of small volumes of liquid reagents at the point-of-testing; these include liquid-handling robots and micro- and nano-fluidic channels combined with pumps and syringes and devices in which the liquids are driven by capillary action.

The use of fluid spacers to separate aliquots of fluid in a device for delivering fluids from a vessel, in series, to a reaction site is described in WO 2005/072858.

A device for the sequential delivery of flowable reagents from a reagent column in which the individual reagents are segregated by movable partitions slidably supported at the inner surface of a container holding the reagent column is described in US 2003/0039588.

Cartridges for use in chemical and biological assays have also been developed; WO 2008/037995, for example, describes a cartridge system comprising a reagent component in which one or more reagents can be stored and a processing component for processing the reagents in an assay where the waste materials from the assay can be stored in the cartridge to prevent contamination.

There remains, however, a continuing need for the development of a simple, low cost device for delivering a series of liquid reagents, at a controllable rate and with defined volume control, to a single point in order to perform a chemical or biological reaction.

### SUMMARY OF THE INVENTION

In a first aspect, the present invention provides an apparatus for delivering a plurality of liquid volumes sequentially to a reaction site, the apparatus comprising:

(i) a vessel having a plurality of solid spacers arranged in linear order therein so as to define a series of adjacent voids, separated by individual solid spacers, for receiving said liquid volumes, the spacers being movable within the vessel such that liquid volumes disposed in the individual voids are deliverable sequentially through the outlet port to the reaction site, the vessel being provided in a lateral wall with a plurality of reversibly sealable openings through which the liquid volumes for delivery to the reaction site may be introduced into the voids defined therein; and

(ii) an outlet port.

The invention also provides a method for delivering a plurality of volumes of liquid sequentially to a reaction site using such an apparatus.

In a further aspect, the invention provides a unit comprising an apparatus according to the first aspect of the invention and a reaction site component.

The invention also provides a system for performing a chemical or biological reaction comprising a unit as defined above, actuating means arranged to move the solid spacers through the vessel so as to deliver the liquid volumes sequentially to the reaction site and means for determining the outcome of the reaction at the reaction site.

Also provided in a further aspect is the use of an apparatus or unit as defined above in an assay method.

By means of the invention, multiple liquids can be delivered sequentially, in defined volumes and at a controllable rate, to a reaction site, facilitating the provision of simple, flexible assay methods which may conveniently be performed in an isolated unit, which may be disposable, by the user at the point-of-sampling. Additionally, the construction of the apparatus according to the invention allows liquid reagents for delivery to the reaction site to be stored within the vessel, thereby minimising the risk of contamination and increasing the reliability of the reaction at the reaction site.

### BRIEF DESCRIPTION OF THE DRAWINGS

FIG. 1 shows schematically an apparatus according to the invention and illustrates its principle of operation

FIG. 2 shows schematically an alternative apparatus according to the invention and its principle of operation.

FIG. 3 is a schematic illustration of a disposable unit according to the invention.

FIGS. 4 and 5 show the unit of FIG. 3 with actuator rods in place to control movement of the liquid volumes through the unit.

### DETAILED DESCRIPTION OF THE INVENTION

The present invention relates to an apparatus and method for delivering a plurality of liquid volumes sequentially to a reaction site.

Any liquid necessary for the reaction at the reaction site to occur, including both aqueous and non-aqueous liquids, may suitably be delivered to the reaction site by means of the apparatus according to the invention.

Suitably, the reaction may be any chemical or biological reaction including, for example, chemical or biological assays to determine the presence and/or quantity of proteins, nucleic acids or other chemical or biological markers of diseases in humans or animals and the liquids for delivery to the reaction site may include liquid reagents, solvents or washes for use in such processes.

The plurality of liquid volumes may comprise the same or different liquids depending on the reaction in which they are intended to participate. Where the intended reaction is an immunoassay for determining the presence of an antigen in a sample, for example, then the apparatus according to the invention may be configured so as to deliver sequentially to the assay site an antibody solution, a labelled antibody solution and optionally rinse solutions, as appropriate. The ability to deal with multiple analytes in one immunoassay using an apparatus according to the invention renders this particularly useful for use in human and animal health applications where multiple biomarkers need to be accurately quantified.

The apparatus according to the invention may be used to deliver any number of liquid volumes sequentially to a reaction site, constrained only by the dimensions of the vessel in which the liquid-containing voids are present.

It will be appreciated that a vessel for use in the apparatus according to the invention may be of any dimensions provided that it has an essentially constant cross-section such that the solid spacers disposed therein to define voids for receiving the liquid volumes are moveable within the vessel in order to deliver the liquid volumes contained in the individual voids sequentially to the vessel outlet port.

Typically, the vessel may be made of any material which is capable of retaining liquids and which does not interact with the liquids to be retained. Any inert material conventional in the art such as plastics, metal, glass or composite materials may suitably be used. Suitable plastics materials conventional in the art which may be used include, for example, polypropylene, polystyrene, acrylonitrile butadiene styrene, polyethylene or polycarbonate.

In one particular embodiment, the vessel for use in the apparatus according to the invention comprises a tube.

The tube may be open at both ends. Conveniently, however, an end of the tube is at least partially closed so as to prevent the spacers from being expelled from the tube in use. The end of the tube may conveniently be at least partially closed by any closing means which are capable of preventing the spacers from being expelled from the tube but which preferably allow air trapped in the vessel to escape. Examples of suitable closing means include caps or stoppers, which may conveniently be provided with air vents, or washers.

Conveniently, a tube will have an inner diameter of 4 mm to 10 mm and typically a length of 5 cm to 12 cm. In one particular embodiment, a tube having a constant inner diameter may be used.

The plurality of solid spacers are arranged in linear order within the vessel such that, together with the vessel walls, they define a series of adjacent voids, separated by individual solid spacers, for receiving the plurality of liquid volumes to be delivered to the reaction site.

For any given application, the number of solid spacers included in the apparatus will depend on the number of liquids which are required to be delivered to the reaction site in order to complete the intended reaction sequence. The size of the individual voids formed by the arrangement of solid spacers within the vessel will govern the volume of liquid deliverable therefrom and may be chosen such that the volume of liquid required to allow the intended reaction to be carried out at the reaction site can be delivered to the reaction site.

The solid spacers prevent the liquid volumes from contacting each other in the vessel prior to their sequential delivery to the reaction site from the apparatus according to the invention. It will be appreciated that in order to maintain the individual liquid volumes separate from each other, the dimensions and shape of the solid spacers will suitably be chosen so as to fill the cross-section of the vessel in which they are located.

The solid spacers may suitably be made of any material which is impermeable to, and does not react with, the liquids to be delivered. Suitable materials which may be used include rubber, silicone materials, plastic, metal, glass or composite materials.

The volume of the voids defined by the solid spacers and the walls of the vessel will be proportional to the spacing between the spacers and thus may be varied by dependent on the spacing chosen.

Typically, voids defining liquid-containing volumes of at least 100 nL are formed, suitably 1-10,000 microlitres.

Conveniently, the plurality of solid spacers will be inserted into the vessel, such as a tube, through an open end and each individual spacer located at its desired position by means of locating means such as pegs of set lengths. This process may be automated using motorised actuators to position the spacers.

In one embodiment, the spacers, locating means and vessel may be provided in the form of a kit for assembly by the user at the point of use

The vessel is provided in a lateral wall with a plurality of reversibly sealable openings through which the liquid volumes for sequential delivery to the reaction site may be introduced into the voids defined by the vessel walls and solid spacers.

It will be appreciated that the location of each such opening will be chosen such that it corresponds to the location of an individual void formed within the vessel, enabling the liquid volumes to be introduced into the apparatus in a pre-determined order for subsequent delivery to the reaction site.

The liquid volumes may be introduced into the individual voids using liquid filling techniques conventional in the art. Conveniently, all of the solutions required for the assay may be filled into the separate voids in the vessel at the same time, for example using conventional automatic liquid filling apparatus such as a multi-line filler. In one embodiment, two (or more) such openings are provided in the lateral wall of the vessel at a position corresponding to that of a void formed within the vessel. This is advantageous to facilitate vacuum filling of liquid into the void.

The provision of openings in the lateral wall of the vessel through which the liquid volumes for sequential delivery to the reaction site may be introduced into the individual voids represents a significant advantage for the apparatus according to the invention. In the device described in US 2003/0039588, for example, no such filling openings are provided and the container must be filled through one end of the container tube by sequentially loading the spacer, then the solution, followed by further spacers and solutions individually as required. This method of filling the vessel is much less satisfactory as it is difficult to accurately and reproducibly introduce precise volumes of the individual solutions required and the method is also much less amenable to automation.

Filling the vessel by introducing the liquid volumes separately into their respective voids, as in the present invention, has the additional advantage of avoiding the risk of contamination which can occur when individual solution components are moved through the vessel. This represents a significant drawback for conventional sequential filling operations such as are employed in the device described in US 2003/0039588.

Not only does the provision of lateral reversibly sealable openings in the wall of the vessel of the present apparatus facilitate loading of the liquid volumes for delivery but it is also important for enabling effective operation by facilitating the positioning of the spacers within the vessel. Where the vessel does not have lateral openings, air trapped between consecutive spacers will become compressed and will tend to force the spacers out of position, meaning that precise and reproducible volumes are difficult to achieve; by providing lateral openings, however, the present apparatus allows air trapped between adjacent spacers to be displaced, thereby allowing the spacers to remain in their intended positions during the filling operation.

Suitable sealing means for sealing the opening include caps or pegs and may be provided as part of the apparatus itself or together with the apparatus for assembly by the user at the point of use.

Suitably the liquid volumes may be disposed in the voids by any conventional technique including manual techniques such as micropipetting or automated techniques such as vacuum filling and automated liquid dispensing.

In one embodiment, the appropriate volumes of the desired liquids may be disposed in the voids defined in the vessel and the vessel sealed so as to store the liquid volumes for later delivery to the reaction site.

By separating the volumes of liquid by means of solid spacers which are movable within the vessel, the respective individual volumes may be delivered to the reaction site in a predetermined sequence and contact between the individual liquid volumes may be avoided.

If mixing of the individual liquid volumes prior to delivery to the reaction site is desirable, however, then this can be achieved according to another embodiment by providing the apparatus with means for allowing two or more of the voids in which the liquid volumes are disposed to come into fluid communication when the apparatus is in use.

In this embodiment, the means are positioned relative to the solid spacers such that fluid communication between the voids is prevented by one or more of the solid spacers prior to actuation, but upon actuation, the spacers move, allowing fluid communication to be established and the contents of one of more of the connected voids to be driven into the other connected void.

Suitably, the means for allowing two or more of the voids to come into fluid communication comprise a tube connecting the two or more voids together, which tube is preferably located on the exterior of the vessel. The vessel is suitably provided with a reversibly sealable vent hole to allow air from the void into which the contents of the one or more other connected voids are driven to vent as the liquid volumes are mixed. It will be appreciated that the reversibly sealable vent hole will be positioned such that it is not obscured by the solid spacer when the apparatus is actuated.

Mixing individual liquid volumes in the vessel prior to delivery to the reaction site in this way is advantageous where the reaction requires that two or more reagents are mixed together before use, particularly where the reagents are unstable, as it avoids having to prepare a pre-mix having a limited shelf life.

The vessel comprises an outlet port through which in use, liquid volumes disposed in the individual voids are deliverable sequentially to the reaction site.

In one embodiment, the outlet port is provided in a lateral wall of the vessel. In the embodiment where the vessel is a tube, the outlet port is suitably located towards one end of the tube.

In use, the solid spacers are movable along the longitudinal axis of the vessel towards the outlet port, exerting force on the liquid volumes disposed in the voids formed between adjacent spacers so as to drive the series of liquid volumes sequentially through the outlet port to the reaction site.

The liquid volumes disposed in the voids formed in a tube may be delivered sequentially through the outlet port by applying a force to the solid spacer located nearest to that end of the tube furthest from the outlet port.

The force to move the spacers and deliver the liquid volumes disposed in the voids sequentially through the outlet port can be applied by bringing actuating means, such as an actuating rod, into contact with the solid spacer located nearest to that end of the tube furthest from the outlet port. Optionally, the actuating means may be attached to this solid spacer as in a conventional syringe plunger. The force may be applied manually or mechanically, for example using a linear actuator.

Alternatively, pneumatic control can be used to push the spacers through the vessel or vacuum control can be used to pull them through the vessel.

Constant force can be applied so that one liquid follows another or force can be applied intermittently, in a predetermined pattern such that individual phases of the reaction may take place at the reaction site before the next liquid is delivered.

In order to retain the solid spacers within the vessel after the liquid volumes have been dispensed, the outlet port is positioned at a point in the vessel wall that allows sufficient space between the outlet port and the end of the tube to accommodate all, or all but one, of the spacers. Thus, in use each of the spacers may be driven sequentially past the outlet port, allowing the liquid volume behind it to be emptied through the outlet port. The final solid spacer can likewise be driven past the outlet port or it may be driven just as far as the outlet port, so as to empty the liquid volume ahead of it.

Conventional syringe applicators, designed to dispense fluid from one end of the syringe tube in the same direction as the syringe plunger is depressed, are unsuitable for dispensing multiple discrete fluid aliquots. By contrast, the apparatus of the present invention allows multiple discrete liquid volumes to be dispensed whilst the solid spacers are retained within the vessel. The use of fluid spacers in place of the present solid spacers would not be effective; the spacer fluid would itself be dispensed through the outlet port between each desired liquid volume and this could lead to problems with the spacer fluid reacting with the reaction site and/or inhibiting or blocking access of the dispensed liquid volumes to the reaction areas, adversely affecting the reaction to be performed.

In another aspect, the apparatus according to the invention as described above may be combined with a reaction site component where a chemical or biological reaction may be carried out.

The reaction site component comprises a device including a reaction site and may suitably comprise an assay device for determining the presence and/or quantity of proteins, nucleic acids or other chemical or biological markers of diseases or for monitoring a chemical reaction or an industrial process, for example, including an immunoassay, a turbidometric assay, a colorimetric assay, an agglutination assay, a fluorescence or chemiluminescence assay, an optical, electrical or magnetic detection based assay.

The reaction site component may comprise a sample receiving region which is configured to accept a sample for assaying. Alternatively in a preferred embodiment, the sample is placed in the vessel containing the liquid volumes for delivery to the reaction site.

The vessel for delivering the liquid volumes and the reaction site component may suitably be configured to be coupled together so that they are in fluid communication.

In a preferred embodiment, the vessel and reaction site component are integrally connected and in fluid communication.

Conveniently, fluid communication between the vessel and reaction site component is provided by connecting the outlet port of the vessel to the reaction site component. As the vessel and reaction site are in fluid communication, the liquid volumes delivered sequentially from the vessel through the outlet port upon application of force to the spacers will be applied sequentially to the reaction site.

In one embodiment, the unit according to the invention comprises an additional vessel for delivering a further liquid such as a wash solution to the reaction site. This additional vessel and the reaction site component are suitably configured to be coupled together such that they are in fluid communication and are preferably integrally connected. Conveniently, the vessel for delivering the wash solution will be provided with spacers defining a void in which the wash solution may be contained and an outlet port for delivering the wash solution to the reaction site in a similar manner to that described above.

Preferably the unit according to the invention further comprises a vessel for receiving waste liquid from the reaction site in fluid communication with the reaction site component. The vessel for receiving waste liquid and the reaction site component are suitably configured to be coupled together and are preferably integrally connected. Suitably the vessel for receiving waste liquid from the reaction site is reversibly closable, for example by means of a cap or plug.

In one embodiment, the vessel for delivering liquid volumes sequentially and the vessel for delivering the wash solution are pre-loaded prior to supply to the user.

The unit may be provided as a kit for assembly, by the user, at the point of use but in a particular embodiment, the unit according to the invention is provided on a single platform in the form of a disposable cartridge intended for single use.

The outcome of the reaction at the reaction site may be determined by any number of conventional techniques depending on the reaction concerned. Where an optically based detection method is proposed, the reaction site is suitably provided with an optical window to allow light to pass from the reaction site to the optical detector. Suitable optical detection methods which may be used include chemiluminescence, absorption, fluorescence and Raman scattering techniques

A unit as defined above may be combined with actuating means arranged to move the solid spacers through the vessel so as to deliver the liquid volumes sequentially to the reaction site and means for determining the outcome of the reaction at the reaction site in a system for performing a chemical or biological reaction.

The system suitably further comprises means for controlling the actuating means, such as a computer control system, so as to deliver the liquids to the reaction site in a programmed manner. Means for recording and/or displaying the outcome of the reaction, such as a computer display screen, may also suitably be provided.

In use, a unit as described above is loaded into an apparatus adapted to receive it and comprising actuating means arranged to move the solid spacers through the vessel so as to deliver the liquid volumes sequentially to the reaction site and means for determining the outcome of the reaction at the reaction site.

The apparatus suitably further comprises means for controlling the actuating means and means for recording and/or displaying the outcome of the reaction.

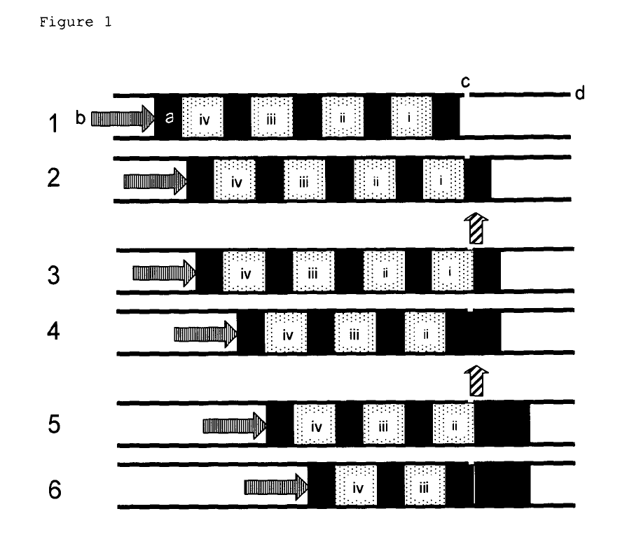

FIG. 1 shows a schematic diagram of an apparatus according to the invention and its principle of operation. The walls of the vessel (which in the embodiment shown is a tube) and solid spacers define multiple voids which can be filled with liquids of interest (labelled i, ii, iii and iv). Stage 1 in FIG. 1 indicates the starting position of such a device as an actuator (b) begins to drive the first spacer (a). Since the liquids are relatively incompressible the actuator has the effect of driving the entire series of liquid volumes (“slugs”) and the spacers along the longitudinal axis of the tube. This continues until stage 2 when the back of the last spacer coincides with the outlet hole (c) in the side of the tube through which the liquids exit. If the actuator pushes the spacers further then slug i is exposed to the outlet hole and the liquid is expelled through the hole (stage 3) until the next to last spacer is driven to touch the last spacer (stage 4) and all of liquid volume i has been expelled. The process is repeated in stages 5 and 6 as liquid volume ii is expelled and so on until all four liquid slugs have been expelled in series.

In this device, the initial spacing of the spacers defines the volume of each of the liquid slugs (which may or may not be the same) and the speed of the actuator linear travel can be used to control the rate of delivery of each of the liquid slugs (which again may or may not be the same).

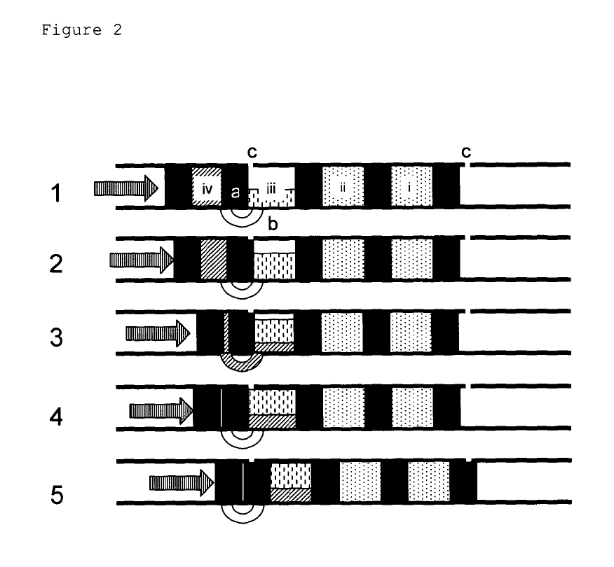

In a modification of the device shown in FIG. 1, two of the liquid chambers defined by the spacers are connected by an external tube so that two of the liquids can be mixed prior to being delivered through the exit hole as shown in FIG. 2.

In FIG. 2, the left hand end of the external pipe is situated at the left hand edge of spacer “a” so it does not initially connect liquids iii and iv. A small vent hole(c) is also included in the chamber containing liquid iii situated the right hand edge of spacer 2. This vent hole may initially be sealed with a removable tab and when open this hole allows the air from the chamber containing liquid iii to vent. In stage 1, liquid iii fills part of the chamber in which it resides which therefore leaves spare volume to accommodate liquid slug iv.

Initially as the actuator pushes the first spacer, it moves and liquid slug iv pushes spacer “a” until the left hand end of the external pipe is exposed at which stage (stage 2) liquid iv is driven along the external pipe into the chamber containing liquid iii. Providing the vent hole is correctly position so that it has not been obscured by spacer “a” then the air from the chamber will vent as liquids iv and iii are mixed. Stage 3 shows the progression of the mixing process and the filling of the chamber which now contains the mixed liquids iv and iii until stage iv when the first spacer meets spacer “a” and spacer “a” is moved to seal the vent hole. At this point the two liquids have been mixed and the operation of the device continues as in FIG. 1 to deliver each of the liquids in turn through the exit hole to the right of the last spacer.

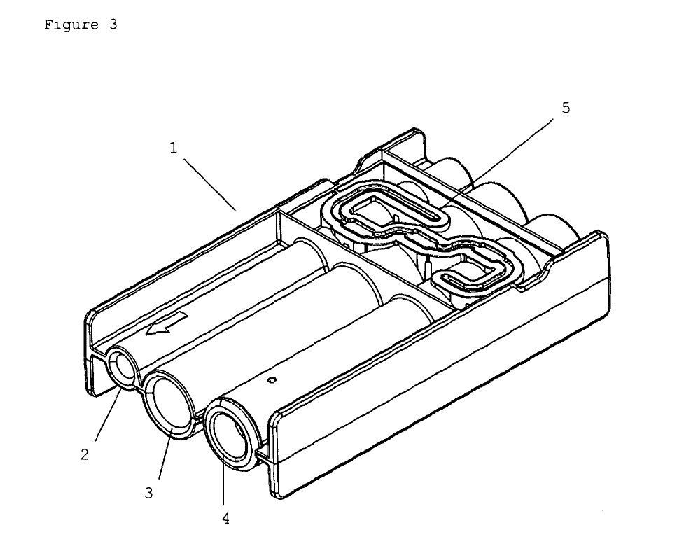

FIG. 3 shows a cartridge (1) according to the invention comprising a reagent tube (2) for containing reagent solutions for an immunoassay, a wash tube (3) for delivering the required wash solution for the immunoassay and a waste collection tube (4). The three tubes are connected by a fluidic channel (5) covered by protein functionalised glass on which the immunoassay occurs.

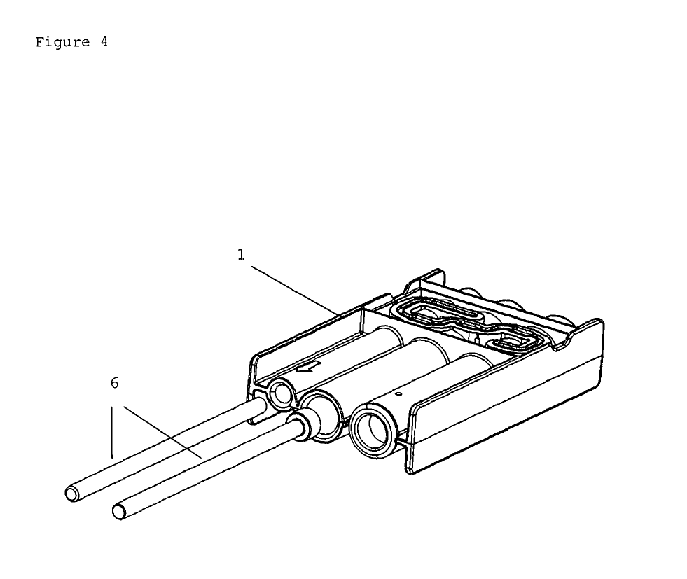

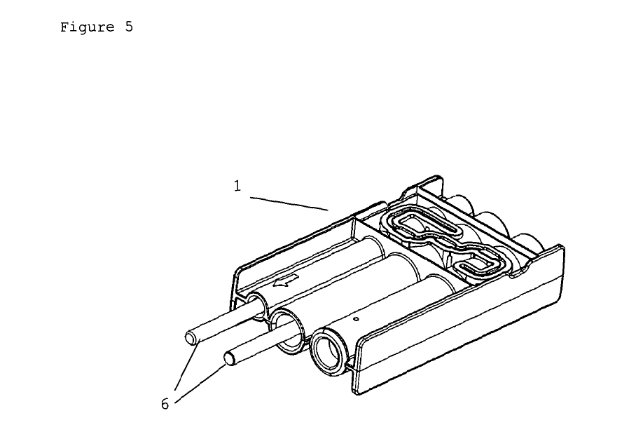

FIG. 4 shows the cartridge of FIG. 3 with actuator rods (6) aligned with the reagent and wash tubes prior to actuation. In FIG. 5, the actuator rods are shown inserted into the cartridge body to control liquid movement through the cartridge by pushing the solid spacers (not shown) in the reagent and wash tubes.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects. Other features of the present invention will become apparent from the following examples.

Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

### EXAMPLES

The invention will now be further illustrated by means of the following non-limiting examples

### Example 1

### Cartridge Preparation

A cartridge was assembled using the following assembly protocol:

Reagent and wash tubes were firstly swabbed with silicon oil to lubricate the tubes. Four reagent bungs and two wash bungs were then inserted into the respective tubes using set length pegs to create three reagent aliquot voids and one wash void in the respective tubes. The appropriate volume of desired reagent and wash solutions were dispensed into the aliquot voids through filling holes in the reagent and wash tubes and filling hole caps inserted into the filling holes to seal them. A waste tube cap is inserted into the end of the waste tube to seal.

Adhesive such as liquid, foam, gel or tape type adhesives is applied to the area surrounding the fluidic channel on the test cartridge and a glass slide with the prepared functionalised reaction areas is placed over the fluidic channel and depressed onto the applied adhesive and allowed to bond accordingly. If required for the particular application a pre-cut piece of opaque masking material such as PVC tape is placed onto the top surface of the glass slide to only expose the reaction areas to the reader unit's detector.

### Example 2

### Assay System

A cartridge prepared as described above in Experiment 1 in conjunction with a reader unit may be used to carry out an automated ELISA test.

The test cartridge is firstly filled with reagents, wash solution, and sample and the glass slide section of the cartridge is functionalised with biomolecules (proteins, antibodies, antigens, nucleotides) specific to the target sample analyte. After inserting the cartridge into a reader unit the user selects the appropriate pre-programmed test and selects to begin the test. The reader unit then drives the test reagents and wash solution through the cartridge by pushing the reagent separating spacers and wash solution spacers in accordance with the predefined programme. As the reagent and wash solutions sequentially pass over the biomolecule functionalised areas on the glass slide the desired assay takes place facilitated by suitable incubation periods for the reagents over the functionalised reaction areas.

The following ELISA assay may be performed:

The cartridge glass reaction areas are pre-functionalised with D. Farinae (dust mite) extract, the reagent tube is loaded with anti-canine IgE HRP antibody and luminol substrate solution and the wash tube is loaded with PBST solution. Diluted canine blood serum is then added into the sample loading port of the cartridge.

As canine blood serum sample is passed over the D. Farinae reaction area ,canine IgE antibodies specific to D. Farinae bind to the funstionalised reaction area during incubation. PBST wash solution is flushed through the cartridge to remove the unbound components of the serum sample. Anti-canine IgE HRP antibody is then flowed through the cartridge and allowed to incubate over the functionalised reaction area, during which the anti-canine IgE HRP antibody binds to the canine IgE antibodies from the canine blood serum sample already bound to the functionalised reaction area. PEST wash solution is then flushed through the cartridge once more to remove the unbound anti-canine IgE HRP antibodies. Finally the luminol substrate solution is flowed over the functionalised reaction area. As this happens the HRP component of the anti-canine IgE HRP antibody undergoes a chemiluminescence reaction with the luminol substrate producing light, and therefore indicating the presence of the analyte (canine IgE antibodies specific to D. Farinae).

1. An apparatus for delivering a plurality of liquid volumes sequentially to a reaction site, the apparatus comprising-:
a vessel having
(i) plurality of solid spacers arranged in linear order therein so as to define a series of adjacent voids, separated by individual solid spacers for receiving said liquid volumes, and
(ii) an outlet port,
the vessel being provided in a lateral wall with a plurality of reversibly sealable openings through which the liquid volumes for delivery to the reaction site may be introduced into the voids defined therein and
the spacers being movable within the vessel such that liquid volumes disposed in the individual voids are deliverable sequentially through the outlet port to the reaction site.

2. An apparatus according to claim 1 wherein the spacers are shaped to fill the cross-section of the vessel.

3. An apparatus according to claim 1 further comprising means for allowing two or more of the voids in which the liquid volumes are disposed to come into fluid communication.

4. An apparatus according to claim 3 wherein the means for allowing two or more of the voids to come into fluid communication comprise a tube connecting the two or more voids together.

5. An apparatus according to claim 3 or claim 4 wherein the means for allowing two or more of the voids to come into fluid communication are positioned relative to the solid spacers such that fluid communication between the voids is prevented by one or more of the solid spacers prior to actuation of the spacers.

6. (canceled)

7. An apparatus according to claim 1 wherein the vessel is a tube.

8. An apparatus according to claim 7 wherein an end of the tube is at least partially closed so as to prevent the spacers from being expelled from the tube in use.

9. (canceled)

10. An apparatus according to claim 7 wherein the outlet port is positioned at a point along the vessel wall that allows sufficient space between the outlet port and the end of the tube to accommodate all, or all but one, of the spacers.

11. A unit comprising an apparatus according to claim 1 and a reaction site component.

12. (canceled)

13. A unit according to claim 11 wherein the reaction site component comprises an assay device.

14. A unit according to claim 11 or claim 13 wherein the vessel for delivering the liquid volumes and the reaction site component are in fluid communication.

15. A unit according to claim 11 or claim 13 wherein the vessel and the reaction site component are integrally connected.

16. A unit according to claim 11 further comprising a vessel for delivering a wash solution to the reaction site and/or a vessel for receiving waste liquid from the reaction site.

17. (canceled)

18. A unit according to claim 11 in the form of a cartridge.

19. A unit according to claim 11 wherein the vessel has a plurality of liquid volumes for delivery to the reaction site disposed therein.

20. A unit according to claim 11 having a wash solution disposed in the wash solution delivery vessel.

21. A system for performing a chemical or biological reaction comprising a unit as defined in claim 11, actuating means arranged to move the solid spacers through the vessel so as to deliver the liquid volumes sequentially to the reaction site and means for determining the outcome of the reaction at the reaction site.

22-23. (canceled)

24. An apparatus adapted to receive a unit according to claim 11 and further comprising actuating means arranged to move the solid spacers through the vessel so as to deliver the liquid volumes sequentially to the reaction site and means for determining the outcome of the reaction at the reaction site.

25. An apparatus according to claim 24 which further comprises means for controlling the actuating means and means for recording and/or displaying the outcome of the reaction.

26. A kit comprising a vessel being provided in a lateral wall thereof with a plurality of reversibly sealable openings and having an outlet port, a plurality of solid spacers and means for locating the spacers within the vessel so as to define a series of voids therein.
